(19) United States
(12) Patent Application Publication
(10) Pub. No.: US 2023/0190175 A1
(43) Pub. Date: Jun. 22, 2023

(54) LIGHTWEIGHT ELECTROENCEPHALOGRAM MONITORING DEVICE WITH SEMI-DRY ELECTRODES

(71) Applicant: MICROSOFT TECHNOLOGY LICENSING, LLC, Redmond, WA (US)

(72) Inventors: Yu-Te WANG, Redmond, WA (US); Ivan Jelev TASHEV, Kirkland, WA (US)

(21) Appl. No.: 17/555,278

(22) Filed: Dec. 17, 2021

(51) Int. Cl. A61B 5/386; A61B 5/291; A61B 5/263; A61B 5/265; G06F 3/01

(52) U.S. Cl. CPC A61B 5/386; A61B 5/291; A61B 5/263; A61B 5/265; G06F 3/015; A61B 5/321

(57) ABSTRACT

A semi-dry electrode combines advantages of wet electrodes and dry electrodes by use of a rotatable ball to apply a conductive gel at the tip of the electrode in a manner similar to how a ballpen applies ink. A reservoir in the semi-dry electrode contains the conductive gel that is applied by the ball to the skin of the user. This creates a thin film of conductive gel at the tip of the semi-dry electrode which reduces impedance and increases the signal-to-noise (SNR) ratio. Directly applying the conductive gel from within the electrode itself reduces mess and improves user convenience. The semi-dry electrode may be used in a lightweight electroencephalography (EEG) monitoring device to detect brain activity. The brain activity may be used as input for a brain-computer interface (BCI).

### BACKGROUND

Electroencephalography (EEG) is a method to record an electrogram of the electrical activity on the scalp that has been shown to represent the macroscopic activity of the surface layer of the brain underneath. It is typically non-invasive, with the electrodes placed along the scalp. EEG measures voltage fluctuations resulting from ionic current within the neurons of the brain. Many systems typically use electrodes, each of which is attached to an individual wire. Some systems use caps or nets into which electrodes are embedded; this is particularly common when high-density arrays of electrodes are needed.

In conventional scalp EEG, the recording is obtained by placing wet electrodes on the scalp with a conductive gel or paste, usually after preparing the scalp area by light abrasion to reduce impedance due to dead skin cells. The conductive gel lowers the impedance between the electrode and the scalp and improves the signal-to-noise ratio (SNR). However, the conductive gel may require a technician to apply and can be difficult to remove from the hair and scalp. Conventional wet electrodes can be impractical outside of medical and research settings.

Some EEG systems use multi-pin dry electrodes without conductive gel that depend upon mechanical contact. The multi-pin dry electrodes penetrate the hair layer and make direct contact with the scalp. Dry electrodes are better suited for wearable devices and informal settings. Dry electrodes avoid the problems associated with the application and removal of conductive gel but typically have higher impedance and a lower SNR than wet electrodes. Thus, it can be difficult to obtain usable signals with dry electrodes. A conductive gel or paste can be applied to the tips of dry electrodes either before use or by careful injection between the hair of the user. This will decrease impedance but has the inconvenience associated with conductive gels.

It would be desirable to have an electrode for use in EEG, and other systems, that provides the convenience and ease-of-use of dry electrodes with the low impedance and high SNR of wet electrodes. Such an electrode and EEG system would have many potential uses including in brain-computer interfaces (BCI). This disclosure is made with respect to these and other considerations.

### SUMMARY

This disclosure provides a semi-dry electrode and a lightweight electroencephalogram monitoring device that incorporates semi-dry electrodes. A semi-dry electrode has a ballpen-like mechanical design with a rotatable ball at the tip of the electrode interfacing with the skin of a user. A reservoir in the semi-dry electrode contains a conductive gel that is applied to the skin of the user as the ball rotates similar to how a ballpen applies ink to paper. As the ball rolls, gravity, capillary action, and/or pressure moves the conductive gel in the reservoir into contact with the ball where it is transferred to the skin. The ball is rotatably retained in the semi-dry electrode by a holder that may be similar to the tip of a ballpen.

The ball may be approximately 1.5-2.5 mm in diameter. The ball is made of a conductive material such as silver/silver chloride. The holder is also made of a conductive material such as silver. In some implementations, the holder and the reservoir may be a single unit formed from the same material. The conductive gel may be any type of electrolyte gel used with wet electrodes such as chloride gel. The semi-dry electrode may be designed for single use, or it may be designed for multiple-time use with a refillable reservoir. In an implementation, the semi-dry electrode may be configured as an elongated cylinder with an external diameter of about 3-5 mm.

A lead conductively connected to the holder carries electrical potential from the semi-dry electrode to circuitry such as an amplifier. In some implementations, the semi-dry electrode itself may contain circuitry such as a pre-amplifier. Electrical potential detected by the semi-dry electrode and processed by circuitry such as the amplifier may be communicated to a computing device. The computing device may record signals from the semi-dry electrode for medical or research applications or process the electrical potential as user input in a BCI system.

The semi-dry electrode may be a component of a wearable EEG monitoring device. The wearable EEG monitoring device may take any one of a variety of form factors such as a head-mounted display (HMD) device, glasses, over-ear headphones, earbuds, watch, activity band, etc. By delivering the electrode gel precisely at the tip of the electrode this design minimizes the amount of electrode gel in contact with the skin of the user and avoids most contact with the user's hair. The presence of electrode gel at the tip of the semi-dry electrode reduces impedance and increases SNR as compared to dry electrodes. The electrode gel is applied as the semi-dry electrode moves relative to the user's skin due to natural movements of the user's body or adjustment of the wearable device. This design saves preparation time, allows the user to apply the semi-dry electrodes themself without assistance from a technician, and simplifies the entire EEG process.

The semi-dry electrodes of this disclosure may be used for applications other than EEG such as detecting electrical activity of the heart with an electrocardiogram (ECG), or muscle activity in electromyography (EMG).

Features and technical benefits other than those explicitly described above will be apparent from a reading of the following Detailed Description and a review of the associated drawings. This Summary is provided to introduce a selection of concepts in a simplified form that are further described below in the Detailed Description. This Summary is not intended to identify key or essential features of the claimed subject matter, nor is it intended to be used as an aid in determining the scope of the claimed subject matter. Furthermore, the claimed subject matter is not limited to implementations that solve any or all disadvantages noted in any part of this disclosure.

### BRIEF DESCRIPTION OF THE DRAWINGS

The Detailed Description is described with reference to the accompanying figures. In the figures, the left-most digit(s) of a reference number identifies the figure in which the reference number first appears. The same reference numbers in different figures indicate similar or identical items. References made to individual items of a plurality of items can use a reference number with a letter of a sequence of letters to refer to each individual item. Generic references to the items may use the specific reference number without the sequence of letters.

FIG. 1 is a cut-away diagram of a semi-dry electrode with an elongated cylindrical configuration.

FIG. 2 is a diagram of a semi-dry electrode that includes a pre-amplifier.

FIG. 3 is a cut-away diagram of a semi-dry electrode with a removable cap providing access to a reservoir for a conductive gel.

FIG. 4 is a cut-away diagram of an alternative configuration of a semi-dry electrode.

FIG. 5 is a cut-away diagram of an alternative configuration of a semi-dry electrode.

FIG. 6 is a cut-away diagram of a semi-dry electrode that contains conductive gel in a balloon.

FIG. 7 is a diagram of a semi-dry electrode applied to the scalp of a user.

FIG. 8 is a schematic diagram of a wearable EEG system.

FIG. 9 is a flow diagram illustrating a method for using the semi-dry electrodes presented herein.

FIG. 10 is a computer architecture diagram illustrating a computing device architecture for a computing device capable of implementing aspects of the techniques and technologies presented herein.

### DETAILED DESCRIPTION

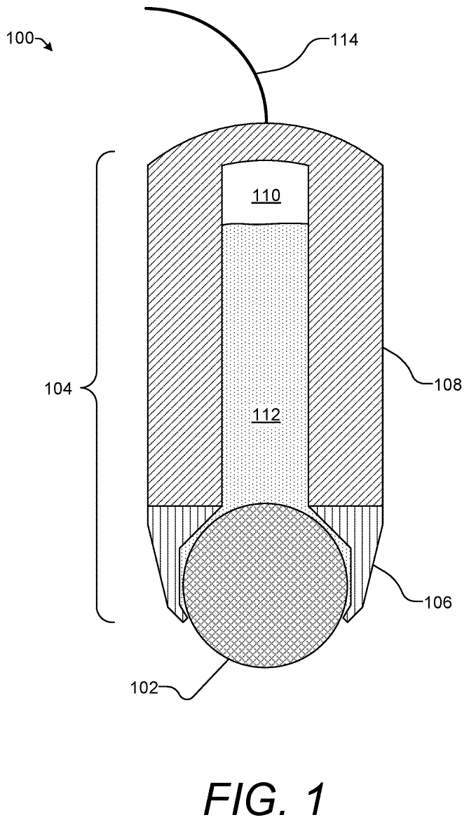

FIG. 1 shows one illustrative configuration of a semi-dry electrode 100 that has at its tip a ball 102 which can freely rotate similar to a ballpen. The ball 102 is rotatably retained in a holder 104. The holder 104 may include a holder tip 106 and a holder body 108. The holder tip 106 is the portion of the holder 104 that is in direct contact with the ball 102 and that retains the ball 102 in the semi-dry electrode 100 while still allowing the ball 102 to freely rotate. The holder 104 may form the body of the semi-dry electrode 100. In particular, the holder body 108 may form the body of the semi-dry electrode 100.

The ball 102 is formed from a conductive material. Many types of conductive materials are known to those of ordinary skill in the art for use with EEG electrodes. Examples of suitable conductive materials include, but are not limited to, conductive metals or metal alloys such as gold, titanium, platinum, silver, copper, tin, nickel, and brass. Other suitable conductive materials include metal compounds such as, but not limited to, iridium-oxide and silver/silver chloride. Further examples of suitable conductive materials include carbon-containing materials such as, but not limited to, graphene and carbon nanotube polydimethylsiloxane. Techniques for forming electrodes from these types of materials are known to those of ordinary skill in the art. For example, a silver/silver chloride electrode may be formed by coating metallic silver with a thin layer of silver chloride. The thin layer of silver chloride may be created either by physically dipping the ball in molten silver chloride, chemically by electroplating the wire in concentrated hydrochloric acid (HCl), or electrochemically by oxidizing the silver at an anode in a chloride solution.

The holder tip 106 is also formed from a conductive material. The holder tip 106 may be made of the same or different conductive material than the ball 102. For example, the holder tip 106 may also be made from a conductive metal or metal alloy, metal compound, or a carbon-containing material. In some implementations, the holder tip 106 may be formed from, for example, gold, silver, or silver/silver chloride. The holder tip 106 may, in some implementations, be coated with a conductive material and have a core of a different material such as a different metal or a plastic.

The holder body 108 may be formed from a conductive material or a nonconductive material. For example, the holder body 108 may be formed from silver, aluminum, copper, stainless steel, metal alloy, plastic, or another material. In some implementations, the entire holder 104 is a single unit formed from the same conductive material. For example, the holder tip 106 and the holder body 108 may be formed from a single piece of silver or other conductive metal.

The semi-dry electrode 100 also includes a reservoir 110 that may be, but is not necessarily, defined by an opening within the holder body 108. The reservoir 110 is in fluid connection with the ball 102 so that conductive gel 112 within the reservoir is in contact with an internal face of the ball 102. The conductive gel 112 may be any type of conductive gel typically used with EEG electrodes. Many types of suitable conductive gel are known to those of ordinary skill in the art including, but not limited to, chloride gel and saline water. The viscosity of the conductive gel 112 may be adjusted using techniques known to those of ordinary skill in the art, such as dilution with a solvent, according to the dimensions and size of the ball 102 in the holder 104. The reservoir 110 may be sealed such that users are not able to add conductive gel 112. A semi-dry electrode 100 intended for a single use may be designed so that the user is not able to add additional conductive gel 112.

As the ball 102 rotates in the holder 104, conductive gel 112 is transferred from the internal face of the ball 102 to an external face that is in contact with the skin of the user. This is similar to how ink within a ball pen is transferred to a sheet of paper. Thus, the external face of the ball 102 will be coated with a thin layer of conductive gel 112 as it moves while in contact with the skin of the user. The layer of conductive gel 112 on the external face of the ball 102 may be refreshed as the ball 102 continues to rotate when the semi-dry electrode 100 moves over the surface of the user's skin.

With current manufacturing techniques, it can be difficult to manufacture balls with diameters smaller than about 2.0 mm. A ball with a diameter greater than 5 mm may have difficulty penetrating the hair on the scalp of a user and contacting the scalp. Thus, in order to have a size that can be reliably manufactured and readily move between the hair to contact the scalp of a user, the ball 102 may have a diameter that is between about 1.0-5.0 mm or in some implementations between about 1.5-2.5 mm. In one implementation, the diameter of the ball 102 may be about 2.0 mm. As used herein, “about,” “around,” “approximately,” and similar referents indicate ±10% of the stated value.

The semi-dry electrode 100 also includes a lead 114 that carries detected electric potential to electronics such as an amplifier. The lead 114 may be a wire formed from a conductive material such as silver, copper, or other material commonly used for electrode leads. The lead 114 is conductively connected to the holder 104. If the entire holder 104 is itself formed from one or more conductive materials, the lead 114 may be connected to any portion of the holder 104. In the example implementation shown in FIG. 1, electrical potential from the skin of a user travels through conductive gel 112 on the external face of the ball 102, the ball 102, the holder tip 106, the holder body 108, and then the lead 114.

In this illustrative configuration, the holder 104 forms an elongated cylinder. The center of the cylinder may be hollow and define the reservoir 110 which contains conductive gel 112. The semi-dry electrode 100 may also have other shapes and configurations. The diameter of the holder 104 may be a few millimeters (e.g., 1-3 mm) greater than the diameter of the ball 102. For example, in some implementations, a diameter of the holder 104 may be about 3-5 mm. A length of the semi-dry electrode 100 may be about 5-30 mm. However, other dimensions and shapes are contemplated.

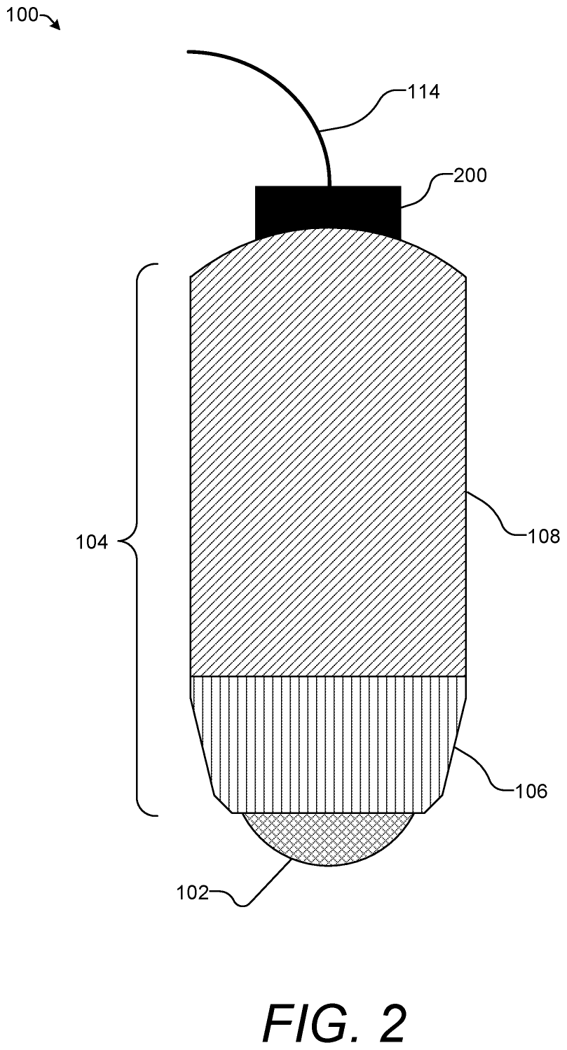

FIG. 2 shows an external view of the semi-dry electrode 100 introduced in FIG. 1. The illustrated components of the same as those in FIG. 1 except for the addition of a preamplifier 200. In some implementations, the preamplifier 200, or other similar electronics, are included in the semi-dry electrode 100. The preamplifier 200 may be any type of conventional preamplifier used on an EEG electrode itself to amplify the detected electrical signal. For example, the preamplifier 200 may be a bio-signal preamplifier. The output of the preamplifier 200 is transmitted through the lead 114 to other electronics such as an Analog-to-Digital Converter (ADC) or to another amplifier. The lead 114 may end with a snap connector configured to couple to an external circuit.

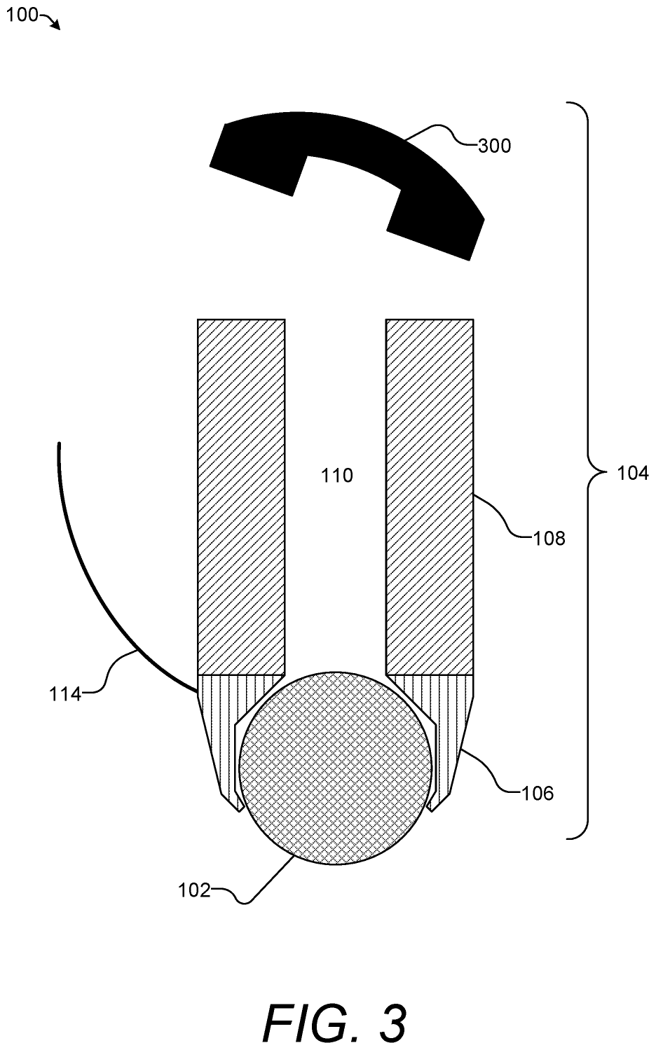

FIG. 3 shows a configuration of the semi-dry electrode 100 with a removable cap 300. The cap 300 may be removed to provide access to the reservoir 110 in order to add or refill the conductive gel 112. Multiple techniques for attaching a cap to a reservoir are known to those of ordinary skill in the art and any suitable structure and type of capping system may be used. The cap 300 may have threads that enable it to screw onto the top of the holder body 108. The cap 300 may also be attached to the holder body 108 by other means such as retaining clips or friction fit. Thus, in some implementations, the semi-dry electrode 100 is provided to the user with an empty reservoir 110. The user can then add conductive gel 112 prior to use of the semi-dry electrode 100. The cap 300 may be made of the same material or different material than the holder body 108.

The implementation illustrated in FIG. 3 shows the lead 114 connected directly to the holder tip 106. If the holder body 108 is formed from a non-conductive material, the lead 114 may be connected to the holder tip 106 to achieve a conductive connection with the holder 104. The lead 114 may be routed along the side of the holder body 108 or through a groove or opening within the holder body 108.

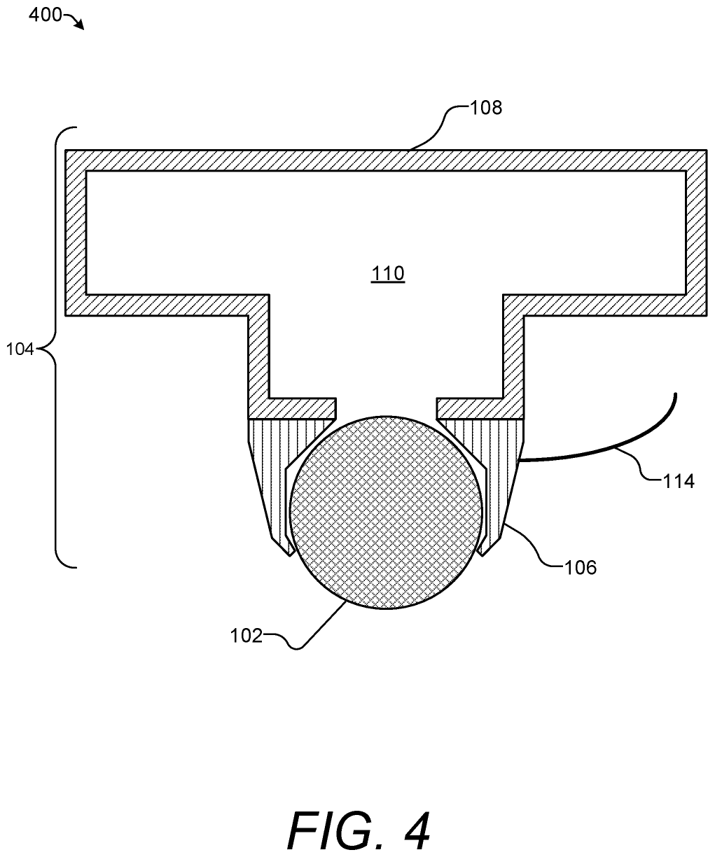

FIG. 4 shows an alternative configuration for a semi-dry electrode 400 in which the holder 104 forms a “T” shape with the ball 102 at the base of the “T.” In this configuration, the reservoir 110 may contain a greater volume of conductive gel 112 than the elongated cylindrical configuration shown in FIGS. 1-3. When viewed from the top, holder body 108 may have a circular, oval, square, rectangular, triangular, or other shape. This configuration is illustrated with the lead 114 connected directly to the holder tip 106. However, if the holder body 108 is also made of conductive material, then the lead 114 may be connected to a location on the holder body.

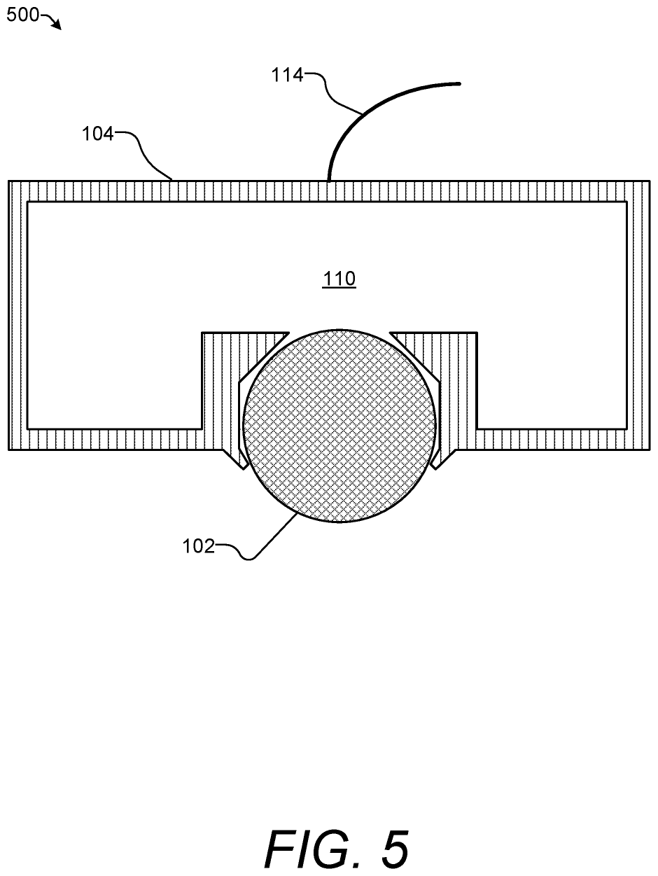

FIG. 5 shows an alternative configuration for a semi-dry electrode 500 with a holder 104 that is a single unit formed from a conductive material. In this configuration, the ball 102 is recessed within the holder 104 such that the bottom of the semi-dry electrode 500 is approximately flat without a tip. This design may be used for areas of the body in which there is less hair and thus less need for the semi-dry electrode to penetrate the hair and make contact with the scalp. For example, this flat design may be used in portions of a HMD that contact the temple of a user, in glasses, or in earphones. This flat design may also be used in a watch or activity band device that contacts the user's wrist where there is less hair than the scalp.

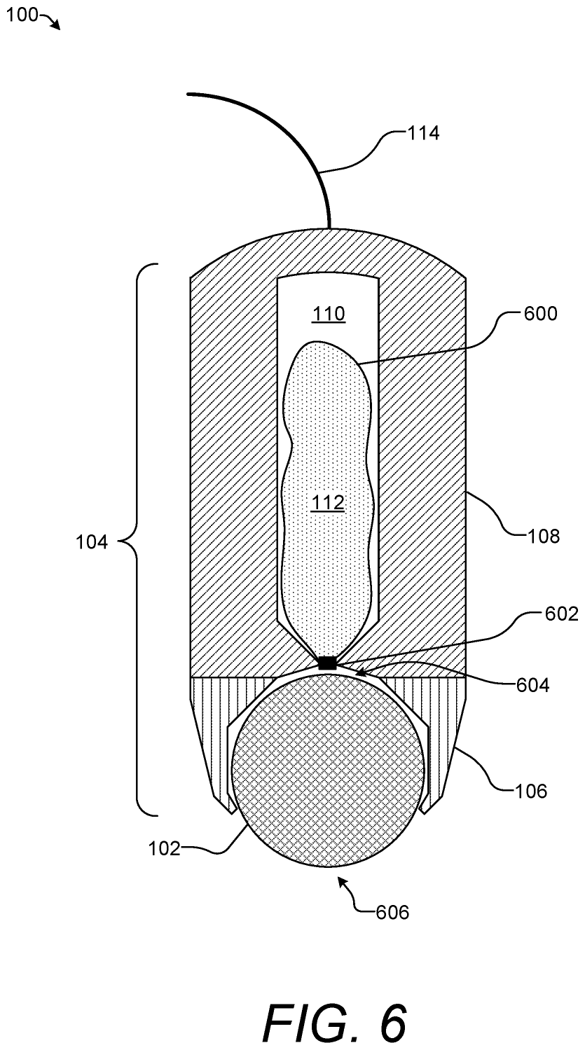

FIG. 6 shows an implementation of the semi-dry electrode 100 in which the conductive gel 112 is contained within a balloon 600. The balloon 600 is formed from an elastomeric material such as, but not limited to, latex or rubber. The balloon 600 is stretched as it is filled with the conductive gel 112. The pressure of the balloon 600 contracting forces the conductive gel 112 out of the reservoir 110 towards the ball 102. The semi-dry electrode 100 may include a filter or valve 602 that regulates the flow of the conductive gel 112 from within the balloon 600 to the internal surface (604) of the ball 102. The external surface (606) of the ball is the portion of the ball that is oriented to contact the skin of the user.

Pressure on the conductive gel 112 provided by the balloon 600 allows the semi-dry electrode 100 to be used in any orientation without relying on gravity to move the conductive gel 112 into contact with the ball 102. Pressure may also be applied to the conductive gel 112 by pressurizing the reservoir 110 through addition of gases at a pressure that is greater than atmospheric. The pressurized gas (e.g., air, nitrogen, etc.) may force the conductive gel 112 through the reservoir 110 into contact with ball 102. Techniques for creating a pressurized chamber that emits a liquid through a rotating ball at any orientation may be adapted from existing techniques used for ballpens such as those described in U.S. Pat. No. 3,285,228.

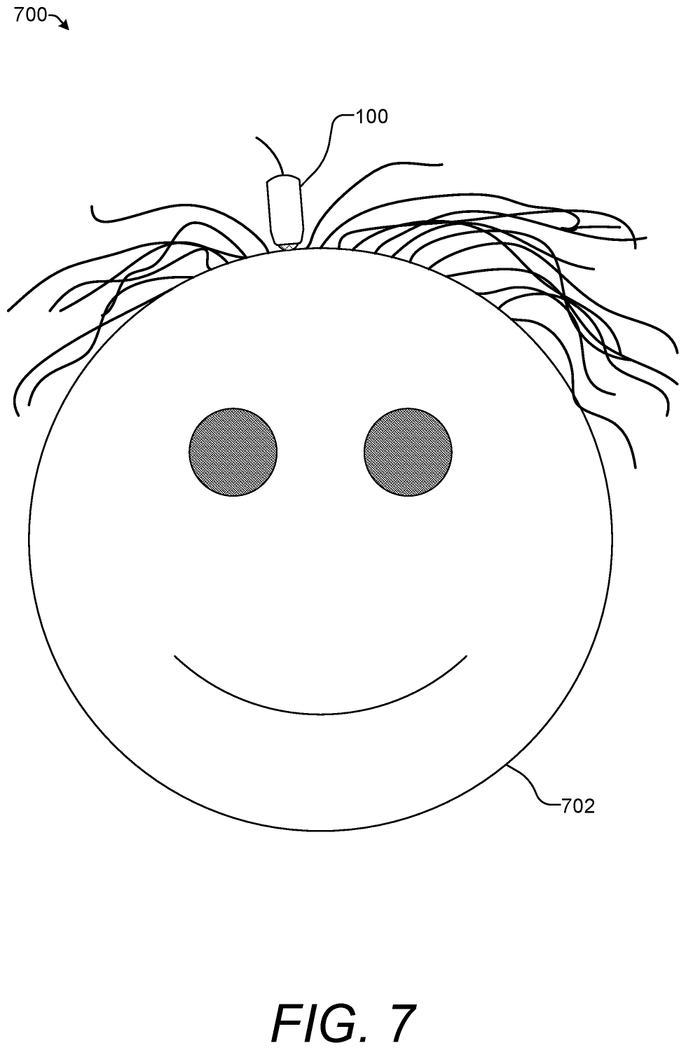

FIG. 7 is a diagram 700 showing use of a semi-dry electrode 100 to detect electrical potential on the scalp of a user 702. The semi-dry electrode 100 may penetrate between the hair of the user 702 to contact the surface of the scalp. The semi-dry electrodes of this disclosure may be used on the scalp or head of the user 702 to detect electrical activity in the brain and generate an EEG. However, the semi-dry electrodes may also be used other portions of the body and to detect different types of electrical signals. For example, the semi-dry electrode 100 may be used for electrocardiography to detect electrical activity of the heart and produce an electrocardiogram (ECG). Similarly, the semi-dry electrode 100 may be used to detect activity of nerve cells that control muscles through electromyography (EMG).

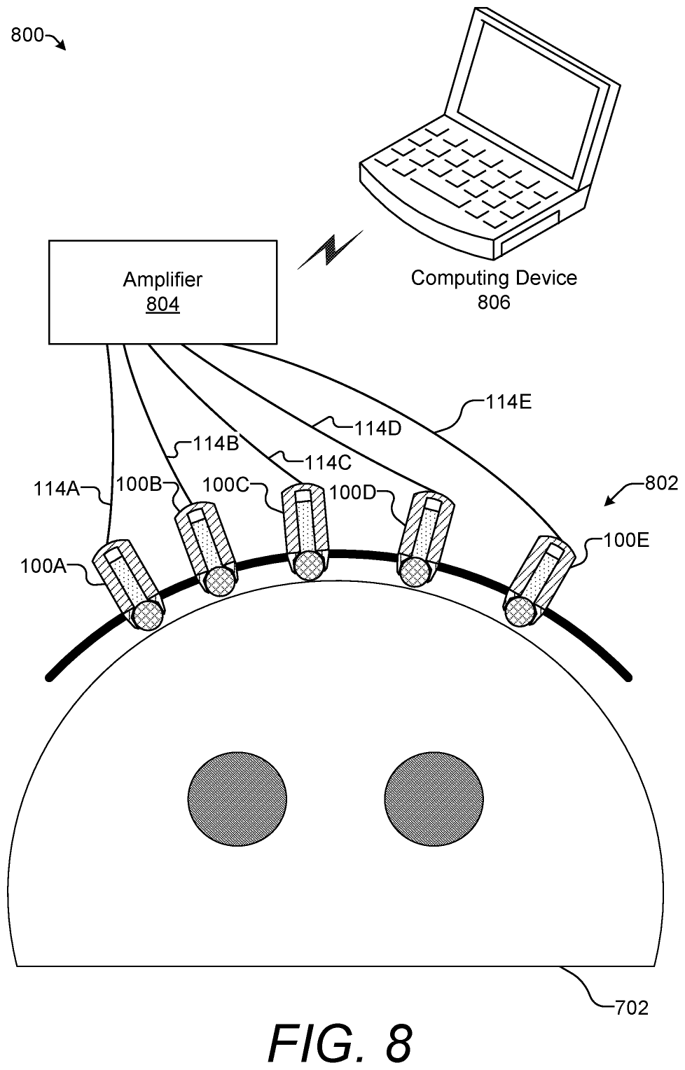

FIG. 8 is a schematic diagram 800 showing the use of a lightweight, wearable EEG monitoring device 802. The wearable EEG monitoring device 802 includes a plurality of semi-dry electrodes 100A-E. Although five semi-dry electrodes 100A-E are shown in this example, the wearable EEG monitoring device 802 may include a greater or lesser number of electrodes. The semi-dry electrodes 100A-E may be implemented as, but are not limited to, any of the configurations of semi-dry electrodes shown in this disclosure. The plurality of semi-dry electrodes 100A-E may include a reference electrode, a common electrode, and a ground electrode as is known in the art of EEG systems. The wearable EEG monitoring device 802 may also include other electronics providing power and/or computing functionality.

The wearable EEG monitoring device 802, with semi-dry electrodes 100A-E, is configured to enable the characterization of brain activity from one or more regions of the user's brain through a non-invasive method. Specifically, each recording electrode can form a channel with the reference electrode. Each channel represents the difference in measured electrical potential between the corresponding recording electrode and the reference electrode. The ground electrode removes electrical potential noise measured globally by each electrode in the device 802.

The wearable EEG monitoring device 802 is illustrated in FIG. 8 as a cap worn over the skull of the user 702. However, the wearable EEG monitoring device 802 may take any number of different form factors and configurations. The wearable EEG monitoring device 802 may be a HMD device such as a virtual reality, augmented reality, or mixed reality headset. The semi-dry electrodes 100 may be included in a band that goes around the temples and back of the head of the user 702, in a strap or band that goes across the top of the head of the user 702, and/or in another location. The wearable EEG monitoring device 802 may also be implemented as glasses that may or may not include a display. When implemented as glasses, the semi-dry electrodes 100 may be included in a portion of the glasses that contacts the skin of the user 702 such as, but not limited to, the nose bridge or the temples above the ears. Additionally, the wearable EEG monitoring device 802 may be implemented as over-ear headphones or earbuds. If implemented as over-ear headphones, the semi-dry electrodes 100 may be included in the earcups of the headphones and contact the user 702 at the skin surrounding the ear. As earbuds, the semi-dry electrodes 100 may contact the inside of the ear. In some implementations, the semi-dry electrodes 100 may be located on other regions of the user's head, such as, on the neck, cheeks, forehead, occipital region, and the like.

Each of the semi-dry electrodes 100A-E is connected by a lead 114A-E to an amplifier 804. The amplifier 804 is an electronic device used to gather and increase the signal amplitude of physiologic electrical activity for output to various sources. It may be an independent unit that is part of the wearable EEG monitoring device 802 or integrated into the electrodes 100. In an implementation, the amplifier is a bio-signal amplifier. The amplifier 804 may include one or more integrated circuits. Signals from the semi-dry electrodes 100A-E to the amplifier 804 may be in the range of about 10 μV to 100 μV, over the frequency range of about 0.1-100 Hz. The strength and frequency of the signals will be different for applications other than EEG. Such applications, the person of ordinary skill in the art can select an appropriate amplifier based on the input provided by the electrodes 100.

The amplifier 804 amplifies the voltage between an active electrode and a reference electrode (typically 1,000-100,000 times, or 60-100 dB of voltage gain). The amplified signal may then be digitized via an ADC (not shown), after being passed through an anti-aliasing filter. Analog-to-digital sampling is typically performed at about 128-1024 Hz in clinical scalp EEG; however, sampling rates of up to 20 kHz may be used. A microcontroller (not shown) in communication with the ADC may function to control generation of a digital EEG signal.

Amplified signals from the amplifier 804 may be communicated to a computing device 806 using an ADC. The computing device 806 may be integrated into the wearable EEG monitoring device 802 or it may be a separate device. For example, the computing device 806 may be a smartphone, smartwatch, tablet, laptop, notebook computer, desktop computer, network accessible computer, or other type of device. The computing device 806 may be communicatively connected with the amplifier 804 via a wired or wireless connection. For example, the amplifier 804 may use radio waves such as Bluetooth® to transmit signals to the computing device 806. The computing device 806 may include EEG software for processing signals received from the amplifier 804 such as EEGLAB (Delorme, Arnaud; Makeig, Scott (2004) “EEGLAB: an open source toolbox for analysis of single-trial EEG dynamics including independent component analysis”. Journal of Neuroscience Methods. Elsevier BV. 134(1): 9-21.) or the Neurophysiological Biomarker Toolbox (available from www.poil.dk/s/nbt-v0-5-1-alpha/874#.VIzD-qbEht1).

Signals received by the computing device 806 may also be used as user input to create a BCI between the wearable EEG monitoring device 802 and the computing device 806. In some implementations, the computing device 806 may have specialized hardware such as OpenBCI which is an open-source brain-computer interface platform (see openbci.com). Additional applications besides conventional EEG and BCI include, but are not limited to, monitoring of the brain state (cognitive load, stress, attention, fatigue due to long hours of working) and monitoring of biological wellbeing.

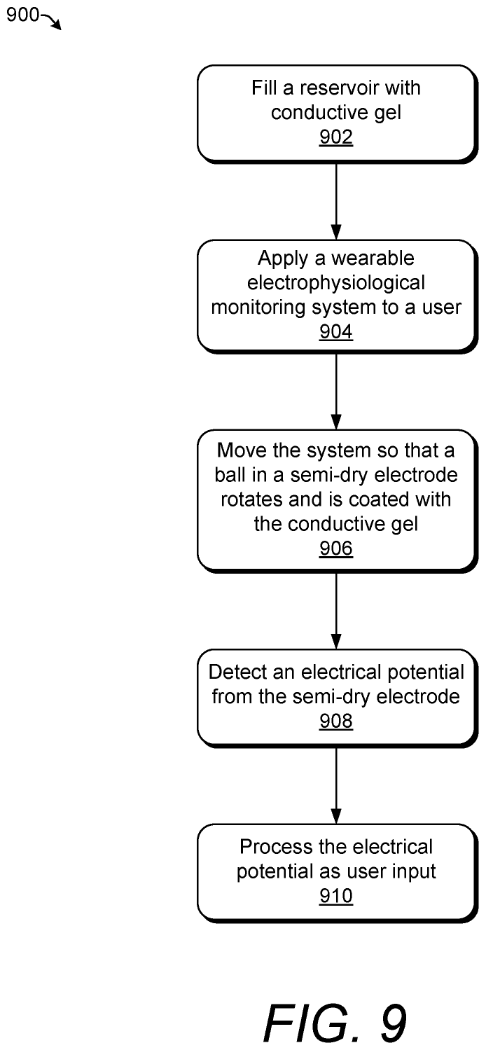

FIG. 9 is a flow diagram illustrating a method 900 for detecting electrical potential on the skin of the user with a semi-dry electrode. Method 900 may be implemented with any of the semi-dry electrodes shown in FIGS. 1-6 or the system shown in FIGS. 7 and 8.

At operation 902, a reservoir of a semi-dry electrode is filled with a conductive gel. This optional step may be omitted if the reservoir is provided to the user filled with conductive gel. In implementation, the filling is done by opening a valve or cap on the reservoir in the semi-dry electrode as shown in FIG. 3 and filling the reservoir with the conductive gel. The conductive gel may be any type of conductive gel suitable for use with electrodes such as, for example, chloride gel or saline solution. The viscosity of the electrode gel may be adjusted based on the design and size of the semi-dry electrode. The semi-dry electrode may be one of a plurality of electrodes included in a wearable electrophysiological monitoring system such as, for example, the wearable EEG monitoring device shown in FIG. 8. The conductive gel may be added to the reservoir of the semi-dry electrode before the wearable electrophysiological monitoring system is applied to the user. This may be done by the user or by another person.

At operation 904, the wearable electrophysiological monitoring system that contains one or more semi-dry electrodes is applied to the user. The wearable electrophysiological monitoring system may take any number of different form factors and configurations. For example, the electrophysiological monitoring system may be an HMD such as a virtual reality, augmented reality, or mixed reality headset. As a further example, the electrophysiological monitoring system may be implemented as glasses, over-ear headphones, earbuds, watch, activity band, etc. The user may be able to apply the wearable electrophysiological monitoring system themselves without assistance from a technician because there is no need to separately apply conductive gel.

At operation 906, the wearable electrophysiological monitoring system is moved while in contact with the skin of the user. The wearable electrophysiological monitoring system may move naturally as the user's body moves. Also, the user may deliberately reposition, adjust, or shift the position of the wearable electrophysiological monitoring system relative to their skin. This movement causes a ball in the semi-dry electrode to rotate within a holder which transfers the conductive gel from the reservoir to the external surface of the ball touching the skin of the user. Presence of the conductive gel on the external surface of the ball reduces impedance and increases the SNR of the semi-dry electrode.

When the wearable electrophysiological monitoring system is first applied to the user, the external surface of the ball touching the skin of the user may be dry and not coated with conductive gel. This may reduce contact of the conductive gel with the user's hair or other portions of the user's skin while donning the electrophysiological monitoring system. However, because the external surface of the ball is not coated with conductive gel when initially worn in this implementation, movement of the electrophysiological monitoring system relative to the skin of the user may be necessary to rotate the ball and apply the conductive gel. The semi-dry electrode and the electrophysiological monitoring system will work even if an external surface of the ball semi-dry electrode is not coated with the conductive gel. However, there may be higher levels of impedance and lower SNR.

At operation 908, an electrical potential is detected from the semi-dry electrode. The electrical potential may be detected between the semi-dry electrode and a reference electrode. The electrical potential can be detected from the skin of the user using any number of techniques and hardware such as circuitry amplifiers known to those of ordinary skill in the art. The electrical potential may be used as an input to EEG, ECG, EMG, or any other technique that measures electrical potential on the skin of a user.

At operation 910, the electrical potential may be processed as user input by a computing device such as, for example, the computing device 806 shown in FIG. 8. However, in some implementations the electrical potential is used in a conventional EEG, ECG, EMG, or other system for detecting bio-electrical activity of the user without being process is user input to a computing device. Thus, operation 910 may not be performed in all implementations. If the electrical potential detected at the semi-dry electrode is from the scalp of the user indicating brain activity, then the electrophysiological monitoring system can be used to implement a BCI by controlling aspects of the operation of the computing device.

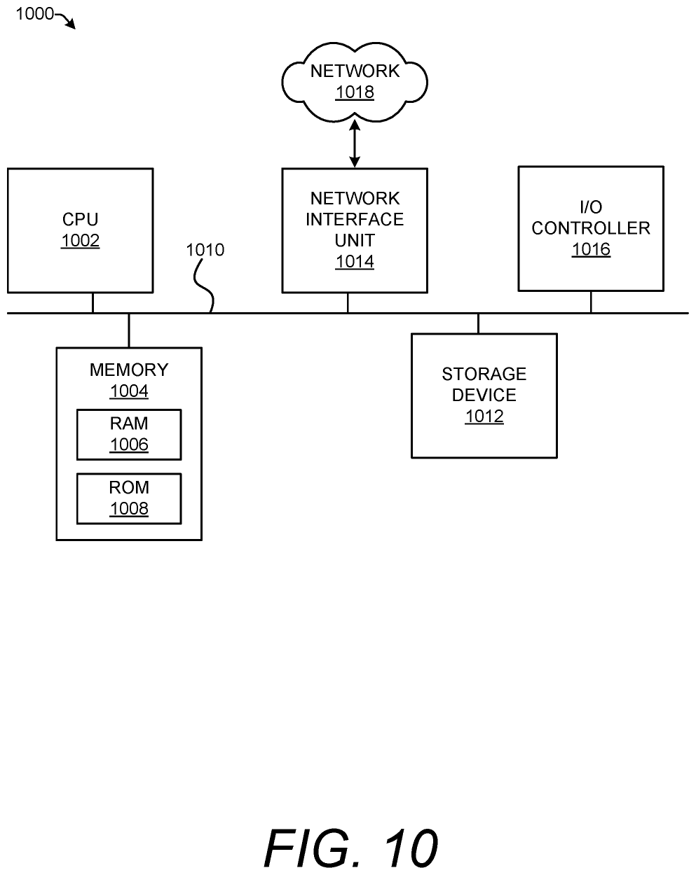

FIG. 10 shows details of an example computer architecture for a computer capable of executing the techniques disclosed herein. Thus, the computer architecture 1000 illustrated in FIG. 10 illustrates an architecture for a portable device, a hand-held device, a wearable device, a desktop device, or network accessible computers, or any other types of computing device(s) suitable for implementing the functionality described herein. Examples of portable devices include, but are not limited to, laptop computers, notebook computers, tablets, and vehicle-mounted computing devices. Examples of hand-held devices include, but are not limited to, smartphones and media players. Examples of wearable devices include, but are not limited to, smartwatches, activity bands, glasses, headphones, earbuds, and HMDs. Examples of network accessible computers include, but are not limited to, server computers and cloud computing systems. The computer architecture 1000 may be utilized to execute any aspects of the computer readable instructions presented herein.

The computer architecture 1000 illustrated in FIG. 10 includes a central processing unit 1002 (“CPU”), a system memory 1004, including a random-access memory 1006 (“RAM”) and a read-only memory (“ROM”) 1008, and a system bus 1010 that couples the memory 1004 to the CPU 1002. A basic input/output system containing the basic routines that help to transfer information between elements within the computer architecture 1000, such as during startup, is stored in the ROM 1008.

Communication media includes computer readable instructions, data structures, program modules, or other data in a modulated data signal such as a carrier wave or other transport mechanism and includes any delivery media. The term “modulated data signal” means a signal that has one or more of its characteristics changed or set in a manner so as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer-readable media.

By way of example, and not limitation, computer storage media may include volatile and non-volatile, removable and non-removable media implemented in any method or technology for storage of information such as computer-readable instructions, data structures, program modules or other data. For example, computer media includes, but is not limited to, RAM, ROM, EPROM, EEPROM, flash memory or other solid state memory technology, CD-ROM, digital versatile disks (“DVD”), HD-DVD, BLU-RAY, or other optical storage, magnetic cassettes, magnetic tape, magnetic disk storage or other magnetic storage devices, or any other medium which can be used to store the desired information, and which can be accessed by the computer architecture 1000. For purposes of the claims, the phrase “computer storage medium,” “computer-readable storage medium,” or “computer-readable medium,” and variations thereof, does not include waves, signals, and/or other transitory and/or intangible communication media, per se.

According to various techniques, the computer architecture 1000 may operate in a networked environment using logical connections to remote computers through a network 1018 and/or another network (not shown). The computer architecture 1000 may connect to the network 1018 through a network interface unit 1014 connected to the bus 1010. It should be appreciated that the network interface unit 1014 also may be utilized to connect to other types of networks and remote computer systems. The computer architecture 1000 also may include an input/output controller 1016 for receiving and processing input from a number of other devices, including a keyboard, mouse, or electronic stylus (not shown in FIG. 10). Similarly, the input/output controller 1016 may provide output to a display screen, a printer, or other type of output device (also not shown in FIG. 10).

It should be appreciated that the computer readable instructions described herein may, when loaded into the CPU 1002 and executed, transform the CPU 1002 and the overall computer architecture 1000 from a general-purpose computing system into a special-purpose computing system customized to facilitate the functionality presented herein. The CPU 1002 may be constructed from any number of transistors or other discrete circuit elements, which may individually or collectively assume any number of states. More specifically, the CPU 1002 may operate as a finite-state machine, in response to computer readable instructions disclosed herein. These computer-executable instructions may transform the CPU 1002 by specifying how the CPU 1002 transitions between states, thereby transforming the transistors or other discrete hardware elements constituting the CPU 1002.

Encoding the computer readable instructions presented herein also may transform the physical structure of the computer-readable media presented herein. The specific transformation of physical structure may depend on various factors, in different implementations of this description. Examples of such factors may include, but are not limited to, the technology used to implement the computer-readable media, whether the computer-readable media is characterized as primary or secondary storage, and the like. For example, if the computer-readable media is implemented as semiconductor-based memory, the computer readable instructions disclosed herein may be encoded on the computer-readable media by transforming the physical state of the semiconductor memory. For example, the computer readable instructions may transform the state of transistors, capacitors, or other discrete circuit elements constituting the semiconductor memory. The computer readable instructions also may transform the physical state of such components in order to store data thereupon.

As another example, the computer-readable media disclosed herein may be implemented using magnetic or optical technology. In such implementations, the computer readable instructions presented herein may transform the physical state of magnetic or optical media, when the computer readable instructions are encoded therein. These transformations may include altering the magnetic characteristics of particular locations within given magnetic media. These transformations also may include altering the physical features or characteristics of particular locations within given optical media, to change the optical characteristics of those locations. Other transformations of physical media are possible without departing from the scope and spirit of the present description, with the foregoing examples provided only to facilitate this discussion.

In light of the above, it should be appreciated that many types of physical transformations take place in the computer architecture 1000 in order to store and execute the computer readable instructions presented herein. It also should be appreciated that the computer architecture 1000 may include other types of computing devices, including hand-held computers, embedded computer systems, personal digital assistants, and other types of computing devices known to those skilled in the art. It is also contemplated that the computer architecture 1000 may not include all of the components shown in FIG. 10, may include other components that are not explicitly shown in FIG. 10, or may utilize an architecture completely different than that shown in FIG. 10.

### Illustrative Implementations

The following clauses described multiple possible implementations for implementing the features described in this disclosure. The various implementations described herein are not limiting nor is every feature from any given implementation required to be present in another implementation. Any two or more of the implementations may be combined together unless context clearly indicates otherwise. As used herein in this document “or” means and/or. For example, “A or B” means A without B, B without A, or A and B. As used herein, “comprising” means including all listed features and potentially including addition of other features that are not listed. “Consisting essentially of” means including the listed features and those additional features that do not materially affect the basic and novel characteristics of the listed features. “Consisting of” means only the listed features to the exclusion of any feature not listed.

Clause 1. In an implementation, this disclosure provides a semi-dry electrode (100) comprising: a reservoir (110) configured to hold a conductive gel (112); a ball (102) formed from a first conductive material; a holder (104) formed from a second conductive material and configured to rotatably retain the ball (102) within the holder (104) and in contact with the reservoir (110); and a lead (114) conductively connected to the holder (104). This implementation provides an advantage of applying conductive gel precisely to the tip of an electrode without the mess or inconvenience associated with having to separately apply conductive gel to electrodes or to the scalp of a user.

Clause 2. In an implementation, this disclosure provides the semi-dry electrode of clause 1, wherein the reservoir contains the conductive gel. This implementation provides an advantage of having the conductive gel readily available and contained within the electrode itself

Clause 3. In an implementation, this disclosure provides the semi-dry electrode of clause 2, wherein the reservoir is sealed. This implementation provides an advantage of convenience and simplicity for the user.

Clause 4. In an implementation, this disclosure provides the semi-dry electrode of clause 3, wherein the conductive gel contained in the reservoir is under pressure. This implementation provides an advantage of configuring the electrode to dispense conductive gel even if oriented at an angle where the ball is above the reservoir so that gravity will not pull the conductive gel towards the ball.

Clause 5. In an implementation, this disclosure provides the semi-dry electrode of any of clauses 1-4, wherein the conductive gel is chloride gel. This implementation provides an advantage of a conductive gel that is safe and a good conductor.

Clause 6. In an implementation, this disclosure provides the semi-dry electrode of any of clauses 1-5, wherein the first conductive material of the ball comprises silver/silver chloride. This implementation provides an advantage of using a conductive material well-suited for electrodes.

Clause 7. In an implementation, this disclosure provides the semi-dry electrode of any of clauses 1-6, wherein a diameter of the ball is about 1.5-2.5 mm, 1.5 mm, 2.0 mm, or 2.5 mm. This implementation provides an advantage of an electrode size that can penetrate hair on the scalp of user yet large enough to be easily manufactured with current techniques.

Clause 8. In an implementation, this disclosure provides the semi-dry electrode of any of clauses 1-7, wherein the second conductive material of the holder comprises silver. This implementation provides an advantage of a material that is conductive, readily available, nonreactive, and moderately priced.

Clause 9. In an implementation, this disclosure provides the semi-dry electrode of any of clauses 1-8, wherein the reservoir and the holder are a single unit formed from the same material. This of implementation provides an advantage of ease of manufacture, simplicity, and durability.

Clause 10. In an implementation, this disclosure provides the semi-dry electrode of clause 9, wherein the holder forms an elongated cylinder with an external diameter of about 3-5 mm, about 3 mm, about 4 mm, or about 5 mm. This implementation provides an advantage of a size that is small enough to be easily placed on the head of user and has sufficient volume to contain a quantity of conductive gel.

Clause 11. In an implementation, this disclosure provides a system for implementing a brain-computer interface (BCI) comprising: a wearable electroencephalography (EEG) monitoring device (802) comprising a plurality of semi-dry electrodes (100A-E), each semi-dry electrode comprising: a reservoir (110) configured to hold a conductive gel (112); a ball (102) formed from a first conductive material; a holder (104) formed from a second conductive material and configured to rotatably retain the ball (102) within the holder (104) and in contact with the reservoir (110); and a lead (114) conductively connected to the holder (104); an amplifier (804) conductively connected to the respective leads (114A-E) of the plurality of semi-dry electrodes (100A-E); and a computing device (806) communicatively connected to the amplifier (804) and configured to process signals received by the amplifier (804) as user input. This implementation provides an advantage of reduced impedance and increased SNR as compared to a system that uses conventional dry electrodes. This implementation provides an advantage of increased cleanliness and greater ease of use as compared to a system that uses conventional wet electrodes.

Clause 12. In an implementation, this disclosure provides the system of clause 11, wherein each of the plurality of semi-dry electrodes comprise a preamplifier (200). This implementation provides an advantage of stronger signals for transmission from the electrode to other components of the system.

Clause 13. In an implementation, this disclosure provides the system of any of clauses 11 or 12, wherein the reservoir contains the conductive gel, the first conductive material of the ball comprises silver/silver chloride, and a diameter of the ball is about 1.5-2.5 millimeters such as 1.5 mm, 2.0 mm, or 2.5 mm. This implementation provides an advantage of ease of use by having the reservoir loaded with conductive gel. This implementation provides an advantage of a conductive material that is well-suited for electrodes applied to the skin of the user. This implementation provides an advantage of an electrode size that easily penetrates the hair on the scalp of user while still being large enough to be readily manufactured with current techniques.

Clause 14. In an implementation, this disclosure provides the system of any of clauses 11-13, wherein the wearable EEG monitoring device comprises a head-mounted display (HMD) device, glasses, headphones, or earbuds. This implementation provides an advantage of user convenience and simplicity by integrating BCI hardware and other functionality into a single wearable device.

Clause 15. In an implementation, this disclosure provides a method of detecting electrical potential on skin of a user, the method comprising: applying a wearable electrophysiological monitoring system to the user (904); moving the wearable electrophysiological monitoring system while in contact with the skin of the user such that a ball in a semi-dry electrode rotates within a holder thereby transferring conductive gel from a reservoir to an external surface (606) of the ball touching the skin of the user (906); and detecting an electrical potential from the semi-dry electrode (908). This implementation provides an advantage of dispensing conductive gel directly from the tips of semi-dry electrodes thereby limiting the need to separately apply electrode gel in a way that may be messy or require assistance from someone other than the user.

Clause 16. In an implementation, this disclosure provides the method of clause 15, wherein the external surface of the ball touching the skin of the user is not coated with the conductive gel when the wearable electrophysiological monitoring system is applied to the user. This implementation provides an advantage of cleanliness by preventing conductive gel from contacting the hair of the user when initially donning the wearable electrophysiological monitoring system.

Clause 17. In an implementation, this disclosure provides the method of any of clauses 15 or 16, further comprising processing the electrical potential as user input by computing device (910). This implementation provides advantage of providing an additional technique for user to control operation of the computing device in place of or in addition to conventional input devices.

Clause 18. In an implementation, this disclosure provides the method of any of clauses 15-17, wherein the electrical potential is an input to electroencephalography (EEG), an electrocardiogram (ECG), or electromyography (EMG). This implementation provides an advantage of using the electrical potential to monitor bioelectrical conditions of a user.

Clause 19. In an implementation, this disclosure provides the method of any of clauses 15-18, wherein the wearable electrophysiological monitoring system comprises a head-mounted display (HMD) device, glasses, over-ear headphones, earbuds, watch, or activity band. This implementation provides an advantage of convenience and simplicity by integrating the wearable electrophysiological monitoring system into a wearable device that provides additional features.

Clause 20. In an implementation, this disclosure provides the method of any of clauses 15-19, further comprising filling the reservoir with conductive gel prior to applying the wearable electrophysiological monitoring system to the user (902). This implementation provides an advantage of allowing the user to select the type of conductive gel as well as making it possible to refill the reservoir when the conductive gel becomes depleted.

### Conclusion

Although the subject matter has been described in language specific to structural features and/or methodological acts, it is to be understood that the subject matter defined in the appended claims is not necessarily limited to the specific features or acts described above. Rather, the specific features and acts are disclosed as example forms of implementing the claims.

The terms “a,” “an,” “the” and similar referents used in the context of describing the invention (especially in the context of the following claims) are to be construed to cover both the singular and the plural unless otherwise indicated herein or clearly contradicted by context. The terms “based on,” “based upon,” and similar referents are to be construed as meaning “based at least in part” which includes being “based in part” and “based in whole,” unless otherwise indicated or clearly contradicted by context.

It should be appreciated that any reference to “first,” “second,” etc. users or other elements within the Summary and/or Detailed Description is not intended to and should not be construed to necessarily correspond to any reference of “first,” “second,” etc. elements of the claims. Rather, any use of “first” and “second” within the Summary, Detailed Description, and/or claims may be used to distinguish between two different instances of the same element (e.g., two different users, two different electrodes, etc.).

Certain embodiments are described herein, including the best mode known to the inventors for carrying out the invention. Of course, variations on these described embodiments will become apparent to those of ordinary skill in the art upon reading the foregoing description. Skilled artisans will know how to employ such variations as appropriate, and the embodiments disclosed herein may be practiced otherwise than specifically described. Accordingly, all modifications and equivalents of the subject matter recited in the claims appended hereto are included within the scope of this disclosure. Moreover, any combination of the above-described elements in all possible variations thereof is encompassed by the invention unless otherwise indicated herein or otherwise clearly contradicted by context.

All patents, patent applications, and references mentioned herein are fully incorporated by reference.

1. A semi-dry electrode comprising:
a reservoir configured to hold a conductive gel;
a ball formed from a first conductive material;
a holder formed from a second conductive material and configured to rotatably retain the ball within the holder and in contact with the reservoir; and
a lead conductively connected to the holder.

2. The semi-dry electrode of claim 1, wherein the reservoir contains the conductive gel.

3. The semi-dry electrode of claim 2, wherein the reservoir is sealed.

4. The semi-dry electrode of claim 3, wherein the conductive gel contained in the reservoir is under pressure.

5. The semi-dry electrode of claim 1, wherein the conductive gel is chloride gel.

6. The semi-dry electrode of claim 1, wherein the first conductive material of the ball comprises silver/silver chloride.

7. The semi-dry electrode of claim 1, wherein a diameter of the ball is about 1.5-2.5 millimeters.

8. The semi-dry electrode of claim 1, wherein the second conductive material of the holder comprises silver.

9. The semi-dry electrode of claim 1, wherein the reservoir and the holder are a single unit formed from the same material.

10. The semi-dry electrode of claim 9, wherein the holder forms an elongated cylinder with an external diameter of about 3-5 mm.

11. A system for implementing a brain-computer interface (BCI) comprising:
a wearable electroencephalography (EEG) monitoring device comprising a plurality of semi-dry electrodes, each semi-dry electrode comprising:
a reservoir configured to hold a conductive gel;
a ball formed from a first conductive material;
a holder formed from a second conductive material and configured to rotatably retain the ball within the holder and in contact with the reservoir; and
a lead conductively connected to the holder;
an amplifier conductively connected to the respective leads of the plurality of semi-dry electrodes; and
a computing device communicatively connected to the amplifier and configured to process signals received by the amplifier as user input.

12. The system of claim 11, wherein each of the plurality of semi-dry electrodes comprise a preamplifier.

13. The system of claim 11, wherein the reservoir contains the conductive gel, the first conductive material of the ball comprises silver/silver chloride, and a diameter of the ball is about 1.5-2.5 millimeters.

14. The system of claim 11, wherein the wearable EEG monitoring device comprises a head-mounted display (HMD) device, glasses, headphones, or earbuds.

15. A method of detecting electrical potential on skin of a user, the method comprising:
applying a wearable electrophysiological monitoring system to the user;
moving the wearable electrophysiological monitoring system while in contact with the skin of the user such that a ball in a semi-dry electrode rotates within a holder thereby transferring conductive gel from a reservoir to an external surface of the ball touching the skin of the user; and
detecting an electrical potential from the semi-dry electrode.

16. The method of claim 15, wherein the external surface of the ball touching the skin of the user is not coated with the conductive gel when the wearable electrophysiological monitoring system is applied to the user.

17. The method of claim 15, further comprising processing the electrical potential as user input by computing device.

18. The method of claim 15, wherein the electrical potential is an input to electroencephalography (EEG), an electrocardiogram (ECG), or electromyography (EMG).

19. The method of claim 15, wherein the wearable electrophysiological monitoring system comprises a head-mounted display (HMD) device, glasses, over-ear headphones, earbuds, watch, or activity band.

20. The method of claim 15, further comprising filling the reservoir with conductive gel prior to applying the wearable electrophysiological monitoring system to the user.
